(19) United States
(12) Patent Application Publication
(10) Pub. No.: US 2019/0365472 A1
(43) Pub. Date: Dec. 5, 2019

(54) Using 3D Imaging and 3D Printing to Occlude a Cerebral Aneurysm

(71) Applicant: Robert A. Connor, St. Paul, MN (US)

(72) Inventor: Robert A. Connor, St. Paul, MN (US)

(73) Assignee: Aneuclose LLC, St. Paul, MN (US)

(21) Appl. No.: 16/541,241

(22) Filed: Aug. 15, 2019

Related U.S. Application Data
(63) Continuation-in-part of application No. 15/865,822, filed on Jan. 9, 2018.
Application No. 15/865,822 is a continuation-in-part of application No. 14/526,600, filed on Oct. 29, 2014, now abandoned.
Application No. 14/526,600 is a continuation-in-part of application No. 12/989,048, filed on Oct. 21, 2010, now Pat. No. 8,974,487.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.

(51) Int. Cl. A61B 34/10; A61B 17/12

(52) U.S. Cl. CPC A61B 34/10; A61B 17/1214; A61B 17/12186; A61B 17/12113; A61B 2017/00017; A61B 17/12163; A61B 2034/105; A61B 2034/104; A61B 17/12172

(57) ABSTRACT

This invention is a method which uses 3D medical imaging to estimate the optimal amount of embolic material: to be inserted into a flexible net, mesh, bag, liner, or stent within a cerebral aneurysm sac in order to optimally occlude the aneurysm; or to be inserted directly into the sac in order to optimally occlude the aneurysm.

### CROSS-REFERENCE TO RELATED APPLICATIONS

This patent application:

(1) claims the priority benefit of U.S. Provisional Patent Application 62/794,609 entitled “Intrasacular Cerebral Aneurysm Occlusion Devices with Multiple Sections and Variable Surface Characteristics” by Robert A. Connor filed on Jan. 19, 2019;

(2) claims the priority benefit of U.S. Provisional Patent Application 62/794,607 entitled “Intrasacular Cerebral Aneurysm Occlusion Devices with Distal-Proximal Stacked Sections and Non-Uniform Surfaces” by Robert A. Connor filed on Jan. 19, 2019;

(3) claims the priority benefit of U.S. Provisional Patent Application 62/720,173 entitled “Using 3D Imaging and 3D Printing to Occlude a Cerebral Aneurysm” by Robert A. Connor filed on Aug. 21, 2018; and

(4) is a continuation in part of U.S. patent application Ser. No. 15/865,822 entitled “Janjua Aneurysm Net with a Resilient Neck-Bridging Portion for Occluding a Cerebral Aneurysm” by Robert A. Connor which was filed on Jan. 9, 2018—which in turn was a continuation in part of U.S. patent application Ser. No. 14/526,600 entitled “Devices and Methods for Occluding a Cerebral Aneurysm” by Robert A. Connor which was filed on Oct. 29, 2014—which in turn was a continuation in part of U.S. patent application Ser. No. 12/989,048 entitled “Aneurysm Occlusion Device” (i.e. the Janjua Aneurysm Net) by Robert A. Connor and Muhammad Tariq Janjua which has a 371 date of Oct. 21, 2010, a filing date of Apr. 24, 2009, and a priority date of May 1, 2008 which is the U.S. national phase filing of PCT/US 2009/002537 entitled “Aneurysm Occlusion Device” by Robert A. Connor and Muhammad Tariq Janjua filed on Apr. 24, 2009 which claimed the priority benefit of U.S. Provisional Patent Application 61/126,047 entitled “Flow of Soft Members into a Net to Embolize an Aneurysm” by Robert A. Connor which received a filing date of May 1, 2008 and claimed the priority benefit of U.S. Provisional Patent Application 61/126,027 entitled “Net Filled with Soft Members to Embolize an Aneurysm” by Robert A. Connor which received a filing date of May 1, 2008; and also claimed the priority benefit of U.S. Provisional Patent Application 61/897,245 entitled “Devices and Methods for Occluding a Cerebral Aneurysm” by Robert A. Connor filed on Oct. 30, 2013.

The entire contents of these related applications are incorporated herein by reference.

### FEDERALLY SPONSORED RESEARCH

Not Applicable

### SEQUENCE LISTING OR PROGRAM

Not applicable

### BACKGROUND

### Field of Invention

This invention relates to methods for occluding a cerebral aneurysm.

### INTRODUCTION

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain.

Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Sadly, even among those who survive, approximately one-half suffer significant and permanent deterioration of brain function.

A cerebral aneurysm can be treated by filling it with embolic material in order to reduce the flow of blood into the aneurysm sac. Once blood flow into and within the sac is reduced, blood within the aneurysm sac can embolize and a wall can form along the neck of the aneurysm, thereby therapeutically isolating the aneurysm sac from the parent vessel. In order to properly fill the aneurysm sac, it is important to insert the appropriate amount of embolic material into the sac. If too little material is inserted, then the sac will be insufficiently occluded and blood flow into the sac continues. If too much material is inserted, then the wall of the sac can be stressed, punctured, or ruptured or the material can protrude into the parent vessel.

### REVIEW OF THE PRIOR ART

Priya et al. (U.S. Patent Application Publication 2018/0092690) discloses patient-specific 3D complex coils and methods of making such coils, including custom fixtures for the manufacture of such coils. Specifically, Priya et al. disclose an innovative system to create 3D representations of one or more aneurysms comprising a computer system which receives images of a blood vessel, enables identification of one or more aneurysms in the images, enables segmentation to isolate the one or more aneurysms, enables creation of one or more 3D aneurysm models based on the segmentation, and enables sending of 3D representation data corresponding to the one or more 3D aneurysm models, wherein the data is used to creation of a 3D representations of the one or more aneurysms. Nonetheless, there remains a need for a method to estimate the optimal amount of embolic material to be inserted into an aneurysm sac in order to occlude the sac optimally.

### SUMMARY OF THE INVENTION

A cerebral aneurysm can be treated by filling it with embolic material in order to reduce the flow of blood into the aneurysm sac. Once blood flow into and within the sac is reduced, blood within the aneurysm sac can embolize and a wall can form along the neck of the aneurysm, thereby therapeutically isolating the aneurysm sac from the parent vessel. In order to properly fill the aneurysm sac, it is important to insert the appropriate amount of embolic material into the sac. If too little material is inserted, then the sac will be insufficiently occluded and blood flow into the sac continues. If too much material is inserted, then the wall of the sac can be stressed, punctured, or ruptured or the material can protrude into the parent vessel. This invention is a method to estimate the optimal amount of material to be inserted into a particular aneurysm sac in order to occlude the sac optimally.

This disclosure specifies a method which uses 3D medical imaging to estimate the optimal amount of embolic material to be inserted into a flexible net, mesh, bag, liner, or stent within an aneurysm sac. This disclosure also specifies a method which uses 3D medical imaging to estimate the optimal amount of embolic material to be inserted directly into an aneurysm sac if a flexible net, mesh, bag, liner, or stent is not used. This disclosure also specifies a method of using 3D imaging to create a custom-shaped net, mesh, bag, liner, or stent for insertion into an aneurysm sac and filling with embolic material.

In an example, this invention can be embodied in a method to occlude a cerebral aneurysm comprising: receiving a 3D image of a cerebral aneurysm; and analyzing the 3D image to estimate an optimal amount of embolic material to be inserted into a flexible net, mesh, bag, liner, or stent within the aneurysm in order to occlude the aneurysm. In an example, estimation of the optimal amount of embolic material can depend on one or more factors selected from the group consisting of: general aneurysm shape or type; aneurysm size; aneurysm location; aneurysm rupture status; type of embolic material; shape of embolic material; size of embolic material; softness and/or compressibility of embolic material; parent vessel shape; parent vessel location; patient demographic information; and patient medical history.

In an example, embolic material can comprise embolic coils or ribbons. In an example, embolic material can comprise hydrogels or other gelatinous material. In an example, embolic material can comprise a string-of-pearls structure (i.e. a plurality of embolic members connected by a string, filament, wire, or micro-chain). In an example, a flow of liquid or gelatinous embolic material can be (automatically) pumped into a flexible net, mesh, bag, liner, or stent until the optimal amount of embolic material has been dispensed. In an example, embolic members can be (automatically) delivered into a flexible net, mesh, bag, liner, or stent until the optimal amount of embolic members has been dispensed.

In an example, this invention can be embodied in a method to occlude a cerebral aneurysm comprising: receiving a 3D image of a cerebral aneurysm; and analyzing the 3D image to estimate an optimal amount of embolic material to be inserted into the aneurysm in order to occlude the aneurysm. In an example, an optimal amount of embolic material can be calculated by estimating the total interior volume of the aneurysm based on the 3D image of the aneurysm. In an example, estimation of the optimal amount of embolic material can depend on one or more factors selected from the group consisting of: general aneurysm shape or type; aneurysm size; aneurysm location; aneurysm rupture status; type of embolic material; shape of embolic material; size of embolic material; softness and/or compressibility of embolic material; parent vessel shape; parent vessel location; patient demographic information; and patient medical history.

In an example, embolic material can comprise embolic coils or ribbons. In an example, embolic material can comprise hydrogels or other gelatinous material. In an example, embolic material can comprise a string-of-pearls structure (i.e. a plurality of embolic members connected by a string, filament, wire, or micro-chain). In an example, a flow of liquid or gelatinous embolic material can be (automatically) pumped into a cerebral aneurysm until the optimal amount of embolic material has been dispensed. In an example, embolic members can be (automatically) delivered into a cerebral aneurysm until the optimal amount of embolic members has been dispensed.

In an example, this invention can be embodied in a method to create a device to occlude a cerebral aneurysm comprising: receiving a 3D image of a cerebral aneurysm; creating a 3D model or 3D mandrel based on the 3D image; and wrapping, weaving, braiding, melting, shrinking, or otherwise conforming wires around the 3D model or 3D mandrel in order to create a custom-shaped convex flexible wire mesh which is configured to be inserted into the aneurysm. In an example, embolic material can be inserted into the custom-shaped convex flexible wire mesh after the mesh has been inserted into the cerebral aneurysm.

### DETAILED DESCRIPTION

In an example, embolic material or members can be inserted into a flexible net, mesh, bag, liner, or stent within a cerebral aneurysm. Insertion of the embolic material or members into the net, mesh, bag, liner, or stent can cause the net mesh, bag, or liner to expand and conform to the walls of the aneurysm sac, thereby occluding the aneurysm. In an example, a method to determine an optimal amount of embolic material or members to be inserted into a flexible net, mesh, bag, liner, or stent within a cerebral aneurysm can comprise: (a) receiving a 3D image of a cerebral aneurysm; and (b) analyzing the 3D image to estimate an optimal amount (or an optimal range of amounts) of embolic material or members to be inserted into a flexible net, mesh, bag, liner, or stent within the aneurysm in order to occlude the aneurysm.

In an example, a method to occlude a cerebral aneurysm can comprise: (a) receiving a 3D image of a cerebral aneurysm; (b) analyzing the 3D image to estimate an optimal amount (or an optimal range of amounts) of embolic material or members to be inserted into a flexible net, mesh, bag, liner, or stent within the aneurysm in order to occlude the aneurysm; and (c) inserting the optimal amount of embolic material or members into the flexible net, mesh, bag, liner, or stent within the aneurysm.

In an example, an in-vivo 3D image of a cerebral aneurysm can be created by a medical imaging method selected from the group consisting of: Computerized Tomography (CT), Computerized Tomography Angiography (CTA), Cone Beam Computed Tomography (CBCT), Conoscopic Holography (CH), Digital Subtraction Angiography (DSA), Magnetic Resonance Angiography (MRA), Magnetic Resonance Imaging (MRI), Maximum Intensity Projection (MIP), Medical Holographic Imaging (MHI), Micro Computerized Tomography (MCT), Positron Emission Tomography (PET), Tuned-Aperture Computed Tomography (TACT), and Ultrasound (U/S). In an example, an in-vivo 3D image of a cerebral aneurysm can be a digital image. In an example, an in-vivo 3D image of a cerebral aneurysm can be a volumetric image. In an example, an in-vivo 3D image can be constructed by digitally merging a plurality of 2D images from different perspectives or at different times. In an example, an in-vivo 3D image of a cerebral aneurysm can be created after injection of contrast media into a person's bloodstream.

In an example, there can be an optimal amount (or an optimal range of amounts) of embolic material or members which should inserted into a flexible net, mesh, bag, liner, or stent within an aneurysm with a particular size and shape in order to occlude that aneurysm most effectively and safely. If the amount of embolic material or members inserted into a flexible net, mesh, bag, liner, or stent is less than this optimal amount, then there may be gaps between the flexible net, mesh, bag, liner, or stent and the walls of the aneurysm which allow blood to continue to flow into the aneurysm. If the amount of embolic material or members inserted into a flexible net, mesh, bag, liner, or stent is greater than this optimal amount, then the flexible net, mesh, bag, liner, or stent may exert too much pressure on the aneurysm walls (potentially causing the aneurysm to rupture); the flexible net, mesh, bag, liner, or stent may protrude out of the aneurysm into the parent vessel; or the flexible net, mesh, bag, liner, or stent may leak embolic material.

An optimal amount (or an optimal range of amounts) of embolic material or members to be inserted into a flexible net, mesh, bag, liner, or stent can be estimated by human judgment. In an example, estimation by human judgment of an optimal amount of embolic material or members to be inserted into a flexible net, mesh, bag, liner, or stent can be done based on medical imaging before an aneurysm occlusion procedure. In an example, estimation by human judgment of an optimal amount of embolic material or members to be inserted into a flexible net, mesh, bag, liner, or stent can be done based on real-time medical imaging during an aneurysm occlusion procedure.

However, an automated process to estimate an optimal amount of embolic material or members, such as a process using computer analysis of digital 3D images of an aneurysm, can be more accurate and quicker than estimation based on human judgment. This can help to reduce errors of under or over injection of embolic material or members and can also help to reduce aneurysm occlusion procedure time. Accordingly, it is desirable to have an accurate method for determining the optimal amount (or an optimal range of amounts) of embolic material or members which should be inserted into a particular aneurysm. This disclosure describes methods for automated estimation of an optimal amount (or optimal amount range) of embolic material or members to be inserted into a flexible net, mesh, bag, liner, or stent in an cerebral aneurysm based on analysis of 3D images of that aneurysm.

In an example, an optimal amount (or an optimal range of amounts) of embolic material or members can be expressed as a volume, especially for a liquid or gelatinous embolic material which is dispensed (into a flexible net, mesh, bag, liner, or stent) in a flow. In an example, an optical amount (or an optimal range of amounts) of embolic material or members can be expressed as a percentage the interior volume of an aneurysm. In an example, an optimal amount (or optimal amount range) of embolic material volume can be calculated in steps comprising: (a) estimating the total interior volume of an aneurysm based on 3D images of the aneurysm; (a) subtracting the volume of the perimeter layer of a flexible net, mesh, bag, liner, or stent which is inserted into the aneurysm in order to calculate a remaining interior volume; and (c) expressing the optimal volume of embolic material to be inserted into the flexible net, mesh, bag, liner, or stent as a percentage of the remaining interior volume.

Total Aneurysm Volume (TAV) of a berry or saccular aneurysm can be defined as the combined interior volumes of the dome and neck of the aneurysm, wherein the proximal boundary of the neck is a portion of a virtual arcuate or planar surface which virtually spans where the wall of the parent vessel should be (or once was) without the aneurysm. The Total Aneurysm Volume (TAV) of a specific cerebral aneurysm can be estimated by computer analysis of a 3D image of that aneurysm. This can apply to wide neck aneurysms as well as narrow neck aneurysms. For aneurysms which are not located at a bifurcation, a virtual arcuate surface which is the proximal edge of a neck can be approximated by a virtual cylinder or a conic section. For aneurysms which are located at a bifurcation, a virtual arcuate surface which is the proximal edge of a neck can be approximated by a virtual hyperbola, paraboloid, or conic section.

In an example, Total Aneurysm Volume (TAV) for a particular aneurysm can be estimated by virtually fitting one or more basic geometric shapes to the interior space of an aneurysm. In an example, TAV can be estimated by the volume of a single basic geometric shape which best fits the interior space of the aneurysm. In an alternative example, TAV can be estimated by the composite volume of multiple basic geometric shapes which together best fit the interior space of an aneurysm. In an example, best fit can be quantitatively measured by measuring the sum of squared deviations between the actual interior space of an aneurysm and the one or more geometric shapes which are used to virtually approximate this interior space. In an example, a basic geometric shape can be selected from the group consisting of: sphere; ovaloid; ellipsoid; beehive; cardioid; conic section; cube; cuboid; cylinder; disk; egg shape; pear shape; apple shape; barrel shape; section of an ellipsoid; frustum; hexahedron; hour glass; glass; spherocylindrical; tapered; kidney shape; section of an ovaloid; polyhedron; pyramid; section of a sphere; torus; and triangular prism. In an alternative example, Total Aneurysm Volume (TAV) can be estimated directly by computerized modeling of the shape of an aneurysm interior without the use of basic geometric shapes.

In an example, an anatomic feature or landmark near an aneurysm which is shown in a 3D image can be used as a fiducial marker to help estimate aneurysm size. In an example, a foreign object which is temporarily inserted into a person's vasculature can be used as a fiducial marker to help estimate aneurysm size. In an example, a laser beam which is projected onto the surface of a person's head can be used as a fiducial marker to help estimate aneurysm size. In an example, the computed distance between a medical imaging device and the interior of a person's head where an aneurysm is located can be used to help estimate aneurysm size. In an example, triangulation of sonic energy can be used to estimate the distance between a medical imaging device and an aneurysm in order to help estimate aneurysm size.

In an example, information from two 3D images of a cerebral aneurysm which are created by two different image modalities can be digitally merged in order to estimate Total Aneurysm Volume (TAV) more accurately than is possible with one image modality alone. In an example, these two different image modalities can be selected from the group consisting of: Computerized Tomography (CT), Computerized Tomography Angiography (CTA), Cone Beam Computed Tomography (CBCT), Conoscopic Holography (CH), Digital Subtraction Angiography (DSA), Magnetic Resonance Angiography (MRA), Magnetic Resonance Imaging (MRI), Maximum Intensity Projection (MIP), Medical Holographic Imaging (MHI), Micro Computerized Tomography (MCT), Positron Emission Tomography (PET), Tuned-Aperture Computed Tomography (TACT), and Ultrasound (U/S).

In an example, a database of aneurysms with different characteristics can be created. In an example, this database can include, at a minimum, images of aneurysms and their Total Aneurysm Volumes (TAVs). In addition to aneurysm images and Total Aneurysm Volumes (TAVs), this database can also include one or more fields selected from the group consisting of: general aneurysm shape or type; aneurysm size; aneurysm location; aneurysm rupture status; type of embolic material; shape of embolic material; size of embolic material; softness and/or compressibility of embolic material; parent vessel shape; parent vessel location; patient demographic information; patient medical history; treatment type; amount of embolic material or members used in treatment; and treatment outcomes. In an example, Total Aneurysm Volume (TAV) for aneurysms in the database can be determined based on actual anatomical measurements, three-dimensional modeling, 3D printing; and/or other methods. In an example, a best match can be made between a new aneurysm with an unknown TAV and an aneurysm with a known TAV in the database. In an example, TAV for the new aneurysm can be estimated by the TAV for the best-matching aneurysm in the database. In an example, the best match can be made using automated pattern recognition, machine learning, a neural network, and/or artificial intelligence.

Net Layer Volume (NLV) can be defined as the volume which is occupied by the perimeter layer of a flexible net, mesh, bag, liner, or stent which is inserted into an aneurysm, which does not include the (hollow or empty) space inside the net, mesh, bag, line, or stent. In an example, NLV can be estimated by the volume of liquid which is displaced by a liquid-porous net, mesh, bag, liner, or stent wherein the liquid is allowed to permeate into the interior of the net, mesh, bag, liner, or stent when it is inserted into the liquid (or vice versa). In another example, NLV can be roughly estimated by the volume of a completely-compacted net, mesh, bag, liner, or stent. In an example, Residual Aneurysm Volume (RAV) can be defined as the Total Aneurysm Volume (TAV) minus the Net Layer Volume (NLV). In other words, RAV=TAV−NLV. In the case of a two part device with a proximal stent in addition to a distal net, such as the Janjua Aneurysm Net plus Bridge (JAN-B) device, the volume of the perimeter layer of the proximal stent can also be subtracted from TAV in order to get RAV.

In an example, an optimal volume (or an optimal range of volumes) of embolic material or members for insertion into flexible net, mesh, bag, liner, or stent can be expressed as an absolute volume. In an example, an optimal volume (or an optimal range of volumes) of embolic material or members for insertion into flexible net, mesh, bag, liner, or stent can be expressed as percentage (or range of percentages) of an aneurysm's Total Aneurysm Volume (TAV). In an example, an optimal range of range of volumes of embolic material (or embolic members) as percentages of an aneurysm's TAV can be within the range of 75% to 95%. In an example, an optimal range of range of volumes of embolic material (or embolic members) as percentages of an aneurysm's TAV can be within the range of 85% to 95%. In an example, an optimal range of range of volumes of embolic material (or embolic members) as percentages of an aneurysm's TAV can be within the range of 75% to 100%. In an example, an optimal range of range of volumes of embolic material (or embolic members) as percentages of an aneurysm's TAV can be within the range of 85% to 100%. In an example, an optimal range of range of volumes of embolic material (or embolic members) as percentages of an aneurysm's TAV can be within the range of 95% to 100%.

In an example, an optimal volume (or an optimal range of volumes) of embolic material or members for insertion into flexible net, mesh, bag, liner, or stent can be expressed as percentage (or range of percentages) of an aneurysm's Residual Aneurysm Volume (RAV). In an example, an optimal range of range of volumes of embolic material (or embolic members) as percentages of an aneurysm's RAV can be within the range of 75% to 95%. In an example, an optimal range of range of volumes of embolic material (or embolic members) as percentages of an aneurysm's RAV can be within the range of 85% to 95%. In an example, an optimal range of range of volumes of embolic material (or embolic members) as percentages of an aneurysm's RAV can be within the range of 75% to 100%. In an example, an optimal range of range of volumes of embolic material (or embolic members) as percentages of an aneurysm's RAV can be within the range of 85% to 100%. In an example, an optimal range of range of volumes of embolic material (or embolic members) as percentages of an aneurysm's RAV can be within the range of 95% to 100%.

In an example, an optimal volume (or an optimal range of volumes) of embolic material or members can depend on aneurysm characteristics or patient demographics. In an example, an optimal volume (or an optimal range of volumes) of embolic material or members can depend on one or more factors selected from the group consisting of: general aneurysm shape or type; aneurysm size; aneurysm location; aneurysm rupture status; type of embolic material; shape of embolic material; size of embolic material; softness and/or compressibility of embolic material; parent vessel shape; parent vessel location; patient demographic information; and patient medical history. In an example, a general (or base case) suggested optimal amount of embolic material can be a given percentage of TAV or RAV, but this general (or base care) percentage can be adjusted based on one or more of these factors.

In an example, an optimal volume of embolic material can depend on aneurysm geometry. In an example, an optimal volume of embolic material for an aneurysm with a complex shape can be lower than an optimal volume of embolic material for an aneurysm with a simpler shape, or vice versa. In an example, an optimal volume of embolic material for an aneurysm which can be satisfactorily modeled by a single 3D geometric shape can be higher than an optimal volume of embolic material for an aneurysm which requires multiple 3D geometric shapes to model it, or vice versa. In an example, an optimal volume of embolic material can depend on aneurysm size. In an example, an optimal volume of embolic material for a larger aneurysm can be higher than an optimal volume of embolic material for a smaller aneurysm, or vice versa. In an example, an optimal volume of embolic material can depend on parent vessel characteristics. In an example, an optimal volume of embolic material for an aneurysm at a bifurcation can be higher than an optimal volume of embolic material for an aneurysm which is not at a bifurcation, or vice versa.

In an example, an optimal volume of embolic material can depend on the level of elasticity or flexibility of the flexible net, mesh, bag, liner, or stent which is used. In an example, an optimal volume of embolic material can be higher for a flexible net, mesh, bag, liner, or stent with a higher level of elasticity or flexibility and can be lower for a flexible net, mesh, bag, liner, or stent with a lower level of elasticity or flexibility, or vice versa. In an example, an optimal volume of embolic material can depend on the size of embolic members used. In an example, an optimal volume of embolic material can be higher when using larger (or longer) embolic members and lower when using smaller (or shorter) embolic members, or vice versa. In an example, an optimal volume of embolic material can depend on the type of embolic material used. In an example, an optimal volume of embolic material can be higher when using softer embolic material and lower when using harder embolic material, or vice versa.

In an example, an optimal amount (or an optimal range of amounts) of embolic material or members can be expressed as a length, especially for a longitudinal embolic member or longitudinal series of embolic members which is dispensed (into a flexible net, mesh, bag, liner, or stent) through a lumen in a linear manner. In an example, an optical amount (or an optimal range of amounts) of embolic material or members can be expressed as a length of one or more embolic members which are longitudinally delivered through a catheter. In an example, an optimal amount (or an optimal range of amounts) of embolic material or members can be expressed as a number, especially for a plurality of separate and/or discrete embolic members which are dispensed (into a flexible net, mesh, bag, liner, or stent) through a lumen. In an example, an optical amount (or an optimal range of amounts) of embolic material or members can be expressed as a number of separate embolic members which are delivered through a catheter.

In an example, embolic material or members inserted into a flexible net, mesh, bag, liner, or stent can be biocompatible yarn or fabric. In an example, embolic material or members inserted into a flexible net, mesh, bag, liner, or stent can be blobs of gel. In an example, embolic material or members inserted into a flexible net, mesh, bag, liner, or stent can be embolic coils. In an example, embolic material or members inserted into a flexible net, mesh, bag, liner, or stent can be embolic gel which solidifies after insertion. In an example, embolic material or members inserted into a flexible net, mesh, bag, liner, or stent can be embolic glue. In an example, embolic material or members inserted into a flexible net, mesh, bag, liner, or stent can be embolic liquid which solidifies after insertion. In an example, embolic material or members inserted into a flexible net, mesh, bag, liner, or stent can be selected from the group consisting of: 2-octyl cyanoacrylate; ethyl-2-cyanoacrylate; methyl 2-cyanoacrylate; and n-butyl cyanoacrylate.

In an example, embolic material or members inserted into a flexible net, mesh, bag, liner, or stent can be fiber strips. In an example, embolic material or members inserted into a flexible net, mesh, bag, liner, or stent can be flexible wires. In an example, embolic material or members inserted into a flexible net, mesh, bag, liner, or stent can be hydrogels. In an example, embolic material or members inserted into a flexible net, mesh, bag, liner, or stent can be mesh ribbon. In an example, embolic material or members inserted into a flexible net, mesh, bag, liner, or stent can be micro-beads. In an example, embolic material or members inserted into a flexible net, mesh, bag, liner, or stent can be microscale mesh spheres.

In an example, embolic material or members inserted into a flexible net, mesh, bag, liner, or stent can be microspheres. In an example, embolic material or members inserted into a flexible net, mesh, bag, liner, or stent can be microsponges. In an example, embolic material or members inserted into a flexible net, mesh, bag, liner, or stent can be stream of paste. In an example, embolic material can comprise a shredded musical score, wherein a person can really have a catchy tune stuck in their head. In an example, embolic material or members can be selected from the group consisting of: biocompatible yarn or fabric; blobs of gel; embolic coils; embolic gel which solidifies after insertion; embolic glue; gummi worms; embolic liquid which solidifies after insertion; fiber strips; flexible wires; hydrogels; mesh ribbon; micro-beads; microscale mesh spheres; microspheres; microsponges; stream of paste; and string-of-pearls embolic structure (e.g. a plurality of embolic members connected by a string, filament, wire, or micro-chain).

In an example, embolic material or members inserted into a flexible net, mesh, bag, liner, or stent can be a string-of-pearls embolic structure (e.g. a plurality of embolic members inserted into a flexible net, mesh, bag, liner, or stent connected by a string, filament, wire, or micro-chain). In an example, a string-of-pearls embolic structure can comprise a plurality of embolic members (e.g. microsponges, microspheres, beads, or hydrogels) which are connected by one or more longitudinal flexible members (e.g. filaments, strings, threads, fibers, sutures, yarns, coils, or wires). In an example, a string-of-pearls embolic structure can comprise a plurality of embolic members (e.g. microsponges, microspheres, beads, or hydrogels) which are each centrally connected by one or more longitudinal flexible members (e.g. filaments, strings, threads, fibers, sutures, yarns, coils, or wires). In an example, embolic members can be connected by a continuous longitudinal member which passes through multiple embolic members. In an example, embolic members can be connected by a plurality of discontinuous longitudinal members which are each attached to embolic members.

In an example, embolic members in a string-of-pearls embolic structure can be uniformly spaced along a longitudinal flexible member which connects them. In an example, there can be a uniform distance between embolic members along a longitudinal flexible member. In an example, this uniform distance can be within a range of 1 micron to 10 microns. In an example, this uniform distance can be within a range of 5 microns to 50 microns. In an example, this uniform distance can be within a range of 20 microns to 200 microns. In an example, this uniform distance can be within a range of 100 microns to 1,000 microns. In an example, embolic members in a string-of-pearls embolic structure can be non-uniformly spaced along a longitudinal member which connects them. In an example, embolic members along a proximal portion of a string-of-pearls embolic structure can be separated by a first average distance, embolic members along a distal portion of a string-of-pearls embolic structure can be separated by a second average distance, and the second average distance can be greater than the first average distance, or vice versa.

In an example, embolic members in a string-of-pearls embolic structure can all have the same size. In an example, this size can be within a range of 1 micron to 10 microns. In an example, this size can be within a range of 5 microns to 50 microns. In an example, this size can be within a range of 20 microns to 200 microns. In an example, this size can be within a range of 100 microns to 1,000 microns. In an example, different embolic members in a string-of-pearls embolic structure can have different sizes. In an example, embolic members along a proximal portion of a string-of-pearls embolic structure can have a first size, embolic members along a distal portion of a string-of-pearls embolic structure can have a second size, and the second size can be greater than the first size, or vice versa.

In an example, embolic members in a string-of-pearls embolic structure can all have the same shape. In an example, this shape can be selected from the group consisting of: sphere or section of a sphere; cylinder; conic section; disk; ellipsoid or section of an ellipsoid; ovaloid or section of an ovaloid; torus; egg-shaped; linear ribbon; undulating ribbon; cube; cuboid; pyramid; triangular prism; hexahedron; and polyhedron. In an example, different embolic members in a string-of-pearls embolic structure can have different shapes. In an example, embolic members along a proximal portion of a string-of-pearls embolic structure can have a first shape and embolic members along a distal portion of a string-of-pearls embolic structure can have a second shape.

In an example, embolic members in a string-of-pearls embolic structure can all be made from the same material. In an example, embolic members along a proximal portion of a string-of-pearls embolic structure can be made from a first material and embolic members along a distal portion of a string-of-pearls embolic structure can be made from a second material. In an example, the first material and the second material can differ in one or more characteristics selected from the group consisting of: adhesion; bioactivity; capacitance; compressibility; electrical conductivity; density; ductility; elasticity; hygroscopy; stiffness; stickiness; viscosity; and Young's modulus.

In an example, a string-of-pearls embolic structure which is inserted into a flexible net, mesh, bag, liner, or stent can comprise a plurality of embolic members (e.g. microsponges, microspheres, beads, or hydrogels) which are connected by one or more longitudinal flexible members (e.g. filaments, strings, threads, fibers, sutures, yarns, coils, or wires), wherein surfaces of the embolic members have microscale (or nanoscale) hook-and-eye structures which cause the embolic members to stick to each other upon contact. This can help to prevent the embolic members from leaking out of the flexible net, mesh, bag, liner, or stent. In an example, the embolic members can be separated from each other during delivery through a lumen so that they do not bunch together and clog the lumen, but can come into engaging contact with each other once they exit the lumen into the flexible net, mesh, bag, liner, or stent.

In an example, embolic members can adhere to each other. In an example, embolic members can adhere to each other after they are inserted into a flexible net, mesh, bag, liner, or stent so that they are less likely to escape out of holes in the flexible net, mesh, bag, liner, or stent. In an example, embolic members can adhere to each other after they are inserted into an aneurysm so that they are less likely to protrude out of the aneurysm into the parent vessel. In an example, embolic members or material can have a first level of adhesion (or stickiness) before they are inserted into an aneurysm and have a second level of adhesion (or stickiness) after they are inserted into the aneurysm, wherein the second level is greater than the first level. In an example, embolic members or material can be changed from a first level of adhesion (or stickiness) to a second level of adhesion (or stickiness) by a means selected from the group consisting of: exposure to blood; exposure to body thermal energy; selective application of a chemical substance by a provider and/or device operator; selective application of electromagnetic energy by a provider and/or device operator; selective application of light energy in a selected wavelength by a provider and/or device operator; and selective application of thermal energy by a provider and/or device operator.

In an example, embolic members or material can be fused, congealed, stuck, or adhered together after insertion into an aneurysm by selective intrasacular application of a chemical substance by a provider and/or device operator. In an example, embolic members or material can be fused, congealed, stuck, or adhered together after insertion by selective intrasacular application of electromagnetic energy by a provider and/or device operator. In an example, embolic members or material can be fused, congealed, stuck, or adhered together after insertion by selective intrasacular application of light energy in a selected wavelength by a provider and/or device operator. In an example, embolic members or material can be fused, congealed, stuck, or adhered together after insertion by selective intrasacular application of thermal energy by a provider and/or device operator.

In an example, the delivery of an optimal amount of embolic material or an optimal number of embolic members can be partially or fully automated based on an amount (or range of amounts) which is estimated using the above methodology. In an example, a device can automatically control the amount of embolic material and/or number of embolic members inserted into a flexible net, mesh, bag, liner, or stent in order to insert an optimal amount of embolic material or an optimal number of embolic members.

In an example, a device can automatically pump a flow of liquid or gelatinous embolic material into a flexible net, mesh, bag, liner, or stent until the optimal amount of embolic material has been dispensed. In an example, a device can automatically push a series of longitudinal embolic members (such as coils) into a flexible net, mesh, bag, liner, or stent until the optimal amount or number of embolic members has been dispensed. In an example, a device can automatically deliver a plurality of embolic members into a flexible net, mesh, bag, liner, or stent until the optimal number of embolic members has been dispensed.

In an example, part of the implementation of the above methods can be done with an embolic delivery control device which measures and controls the insertion of embolic material or members into a flexible net, mesh, bag, liner, or stent within an aneurysm so that the optimal amount of embolic material or members is inserted. In an example, an embolic delivery control device for a longitudinal embolic member or series of longitudinal members (such as coils) can measure and control the length or number of embolic members inserted into a flexible net, mesh, bag, liner, or stent within an aneurysm. In an example, an embolic delivery control device to deliver an optimal length or number of longitudinal embolic members into a flexible net, mesh, bag, liner, or stent within an aneurysm can push a desired length or number of embolic members through a lumen into a flexible net, mesh, bag, liner, or stent within an aneurysm.

In an example, this device can selectively cut, sever, snap, melt, or segment (and detach) an otherwise continuous length of embolic material (such as a coil) after the optimal length of the material has been inserted into a flexible net, mesh, bag, liner, or stent within an aneurysm. In an example, an embolic delivery control device can cut a longitudinal embolic member and detach the severed portion after an optimal length of the longitudinal embolic member has been inserted into a flexible net, mesh, bag, liner, or stent within an aneurysm. In an example, an embolic delivery control device can melt a longitudinal embolic member and detach the severed portion after an optimal length of the longitudinal embolic member has been inserted into a flexible net, mesh, bag, liner, or stent within an aneurysm.

In an example, an embolic delivery control device for a liquid or gelatinous embolic material can measure and control the flow of this embolic material into a flexible net, mesh, bag, liner, or stent within an aneurysm. In an example, an embolic delivery control device for delivering a liquid or gelatinous embolic material can comprise a pump. In an example, an embolic delivery control device for delivering a liquid or gelatinous embolic material can be selected from the group consisting of: axial pump, biochemical pump, biological pump, centrifugal pump, convective pump, diffusion pump, dispensing pump, effervescent pump, elastomeric pump, electrodiffusion pump, electrolytic pump, electromechanical pump, electroosmotic pump, fixed-occlusion peristaltic pump, gravity feed pump, helical pump, hose-type peristaltic pump, hydrolytic pump, In various examples, infusion pump, mechanical screw-type pump, Micro Electrical Mechanical System (MEMS) pump, micro pump, multiple-roller peristaltic pump, osmotic pump, peristaltic pump, piezoelectric pump, pulsatile pump, rotary pump, spring-loaded roller pump, tube-type peristaltic pump, and vapor pressure pump.

In an example, a liquid or gelatinous embolic material inserted into a flexible net, mesh, bag, liner, or stent can be a liquid polymer. In an example, a liquid or gelatinous embolic material inserted into a flexible net, mesh, bag, liner, or stent can be selected from the group consisting of: 2-octyl cyanoacrylate; ethyl-2-cyanoacrylate; methyl 2-cyanoacrylate; and n-butyl cyanoacrylate. In an example, a liquid or gelatinous embolic material inserted into a flexible net, mesh, bag, liner, or stent can be selected from the group consisting of: acrylamide-based hydrogel; acrylic-acid-based hydrogel; agar; alginate-based hydrogel; carboxymethyl cellulose; cellulose; chitin; chitosan; collagen; copolymeric hydrogel; gellan; gum arabic; heparin; homopolymeric hydrogel; hyaluronan; hydrocolloid hydrogel; methyl cellulose; multipolymer interpenetrating polymeric hydrogel; pectin; pluronic-acid-based hydrogel; polyacrylic-acid-based hydrogel; polypeptide-based; polyurethane-based; poly-vinyl-alcohol-based hydrogel; starch; superabsorbent hydrogel; superporous hydrogel; and xanthan

In an example, guidance concerning the optimal amount of embolic material or members can be partially, but not fully, automated. In an example, an embolic delivery control device can track and display the cumulative amount of embolic material or members which is being inserted into a flexible net, mesh, bag, liner, or stent during a procedure. In an example, an embolic delivery control device can notify a provider and/or device operator in real time (e.g. with a visual, auditory, or tactile signal) as the cumulative amount of inserted embolic material or members is approaching the optimal amount. In an example, an embolic delivery control device can notify a provider and/or device operator in real time (e.g. with a visual, auditory, or tactile signal) as the cumulative amount of inserted embolic material or members has reached the optimal amount. Example variations discussed in other portions of this disclosure or in priority-linked disclosures can also be applied to these examples where relevant, but are not repeated here in order to reduce narrative redundancy.

In an example, selected types of embolic material or members (e.g. those which are less likely to protrude out of an aneurysm into a parent vessel) can be delivered directly into an aneurysm sac without the need for a flexible net, mesh, bag, liner, or stent. In such examples, it can be useful to have an automated method for estimating the optimal amount of embolic material or members to be inserted into the aneurysm based on 3D imaging of the aneurysm. In an example, a method to determine an optimal amount of embolic material or members to be inserted into an cerebral aneurysm can comprise: (a) receiving a 3D image of a cerebral aneurysm; and (b) analyzing the 3D image to estimate an optimal amount (or an optimal range of amounts) of embolic material or members to be inserted into the aneurysm in order to occlude the aneurysm.

In an example, a method to occlude a cerebral aneurysm can comprise: (a) receiving a 3D image of a cerebral aneurysm; (b) analyzing the 3D image to estimate an optimal amount (or an optimal range of amounts) of embolic material or members to be inserted into the aneurysm in order to occlude the aneurysm; and (c) inserting the optimal amount of embolic material or members into the aneurysm.

In an example, an in-vivo 3D image of a cerebral aneurysm can be created by a medical imaging method selected from the group consisting of: Computerized Tomography (CT), Computerized Tomography Angiography (CTA), Cone Beam Computed Tomography (CBCT), Conoscopic Holography (CH), Digital Subtraction Angiography (DSA), Magnetic Resonance Angiography (MRA), Magnetic Resonance Imaging (MRI), Maximum Intensity Projection (MIP), Medical Holographic Imaging (MHI), Micro Computerized Tomography (MCT), Positron Emission Tomography (PET), Tuned-Aperture Computed Tomography (TACT), and Ultrasound (U/S). In an example, an in-vivo 3D image of a cerebral aneurysm can be a digital image. In an example, an in-vivo 3D image of a cerebral aneurysm can be a volumetric image. In an example, an in-vivo 3D image can be constructed by digitally merging a plurality of 2D images from different perspectives or at different times. In an example, an in-vivo 3D image of a cerebral aneurysm can be created after injection of contrast media into a person's bloodstream.

In an example, there can be an optimal amount (or an optimal range of amounts) of embolic material or members which should be inserted into a particular aneurysm in order to occlude that aneurysm most effectively. If the amount of embolic material or members inserted into an aneurysm is less than this optimal amount, then gaps may remain inside the aneurysm sac which allow blood to continue to flow into the aneurysm. If the amount of embolic material or members inserted into an aneurysm is greater than this optimal amount, then the accumulated embolic material or members may exert too much pressure on the aneurysm walls or protrude out of the aneurysm into the parent vessel.

An automated (or partially automated) process for estimating the optimal amount of embolic material or members, such as a process using computer analysis of digital 3D images, can be more accurate and quicker than estimation of this amount based on human judgment. An automated (or partially automated) process can help to reduce errors of under or over injection of embolic material or members and can also help to reduce aneurysm occlusion procedure time. Accordingly, it is desirable to have an accurate method for determining the optimal amount (or an optimal range of amounts) of embolic material or members which should be inserted into a particular aneurysm. This disclosure describes methods for (partially) automated estimation of an optimal amount (or optimal amount range) of embolic material or members to be inserted into an aneurysm based on analysis of 3D images of that aneurysm.

In an example, an optimal amount (or an optimal range of amounts) of embolic material or members can be expressed as a volume, especially for a liquid or gelatinous embolic material which is dispensed in a flow. In an example, an optical amount (or an optimal range of amounts) of embolic material or members can be expressed as a percentage the interior volume of an aneurysm.

Total Aneurysm Volume (TAV) of a berry or saccular aneurysm can be defined as the combined interior volumes of the dome and neck of the aneurysm, wherein the proximal boundary of the neck is a portion of a virtual arcuate or planar surface spanning where the wall of the parent vessel should be (or once was) without the aneurysm. Total Aneurysm Volume (TAV) of a specific cerebral aneurysm can be estimated by computer analysis of a 3D image of that aneurysm. This can apply to wide neck aneurysms as well as narrow neck aneurysms. For aneurysms which are not located at a bifurcation, a virtual arcuate surface which is the proximal edge of a neck can be approximated by a virtual cylinder or a conic section. For aneurysms which are located at a bifurcation, a virtual arcuate surface which is the proximal edge of a neck can be approximated by a virtual hyperbola, paraboloid, or conic section.

In an example, Total Aneurysm Volume (TAV) for a particular aneurysm can be estimated by virtually fitting one or more basic geometric shapes to the interior space of an aneurysm. In an example, TAV can be estimated by the volume of a single basic geometric shape which best fits the interior space of the aneurysm. In an example, TAV can be estimated by the composite volume of multiple basic geometric shapes which together best fit the interior space of an aneurysm. In an example, best fit can be quantitatively measured by measuring the sum of squared deviations between the actual interior space of an aneurysm and the one or more geometric shapes which are used to virtually approximate this interior space. In an example, a basic geometric shape can be selected from the group consisting of: beehive; cardioid; conic section; cube; cuboid; cylinder; disk; egg shape; pear shape; apple shape; barrel shape; ellipsoid or section of an ellipsoid; frustum; hexahedron; hour glass; glass; spherocylindrical; tapered; kidney shape; ovaloid or section of an ovaloid; polyhedron; pyramid; sphere or section of a sphere; torus; and triangular prism. In an alternative example, Total Aneurysm Volume (TAV) can be estimated directly by computerized modeling of the shape of an aneurysm interior, without the use of basic geometric shapes.

In an example, an anatomic feature or landmark near an aneurysm which is shown in a 3D image can be used as a fiducial marker to help estimate aneurysm size. In an example, a foreign object which is temporarily inserted into a person's vasculature can be used as a fiducial marker to help estimate aneurysm size. In an example, a laser beam which is projected onto the surface of a person's head can be used as a fiducial marker to help estimate aneurysm size. In an example, the computed distance between a medical imaging device and the interior of a person's head where an aneurysm is located can be used to help estimate aneurysm size. In an example, triangulation of sonic energy can be used to estimate the distance between a medical imaging device and an aneurysm in order to help estimate aneurysm size.

In an example, information from two 3D images of a cerebral aneurysm created by two different image modalities can be digitally merged in order to estimate Total Aneurysm Volume (TAV) more accurately than is possible with one image modality alone. In an example, these two different image modalities can be selected from the group consisting of: Computerized Tomography (CT), Computerized Tomography Angiography (CTA), Cone Beam Computed Tomography (CBCT), Conoscopic Holography (CH), Digital Subtraction Angiography (DSA), Magnetic Resonance Angiography (MRA), Magnetic Resonance Imaging (MRI), Maximum Intensity Projection (MIP), Medical Holographic Imaging (MHI), Micro Computerized Tomography (MCT), Positron Emission Tomography (PET), Tuned-Aperture Computed Tomography (TACT), and Ultrasound (U/S).

In an example, an optimal volume (or an optimal range of volumes) of embolic material or members for insertion into an aneurysm can be expressed as an absolute volume. In an example, an optimal volume (or an optimal range of volumes) of embolic material or members for insertion into an aneurysm can be expressed as percentage (or range of percentages) of an aneurysm's Total Aneurysm Volume (TAV). In an example, an optimal range of range of volumes of embolic material (or embolic members) as percentages of an aneurysm's TAV can be within the range of 75% to 95%. In an example, an optimal range of range of volumes of embolic material (or embolic members) as percentages of an aneurysm's TAV can be within the range of 85% to 95%. In an example, an optimal range of range of volumes of embolic material (or embolic members) as percentages of an aneurysm's TAV can be within the range of 75% to 100%. In an example, an optimal range of range of volumes of embolic material (or embolic members) as percentages of an aneurysm's TAV can be within the range of 85% to 100%. In an example, an optimal range of range of volumes of embolic material (or embolic members) as percentages of an aneurysm's TAV can be within the range of 95% to 100%.

In an example, an optimal volume (or an optimal range of volumes) of embolic material or members can depend on aneurysm characteristics or patient demographics. In an example, an optimal volume (or an optimal range of volumes) of embolic material or members can depend on one or more factors selected from the group consisting of: general aneurysm morphology, shape, or type; aneurysm size; aneurysm location; aneurysm rupture status; type of embolic material; shape of embolic material; size of embolic material; softness and/or compressibility of embolic material; parent vessel shape; parent vessel location; patient demographic information; and patient medical history. In an example, a general (or base case) suggested optimal amount of embolic material can be a given percentage of TAV, but this general (or base care) percentage can be adjusted based on one or more of these factors.

In an example, an optimal volume of embolic material can depend on aneurysm geometry. In an example, an optimal volume of embolic material for an aneurysm with a complex shape can be lower than an optimal volume of embolic material for an aneurysm with a simpler shape, or vice versa. In an example, an optimal volume of embolic material for an aneurysm which can be satisfactorily modeled by a single 3D geometric shape can be higher than an optimal volume of embolic material for an aneurysm which requires multiple 3D geometric shapes to model it, or vice versa. In an example, an optimal volume of embolic material can depend on aneurysm size. In an example, an optimal volume of embolic material for a larger aneurysm can be higher than an optimal volume of embolic material for a smaller aneurysm, or vice versa. In an example, an optimal volume of embolic material can depend on parent vessel characteristics. In an example, an optimal volume of embolic material for an aneurysm at a bifurcation can be higher than an optimal volume of embolic material for an aneurysm which is not at a bifurcation, or vice versa.

In an example, an optimal volume of embolic material can depend on the size of embolic members used. In an example, an optimal volume of embolic material can be higher when using larger (or longer) embolic members and lower when using smaller (or shorter) embolic members, or vice versa. In an example, an optimal volume of embolic material can depend on the type of embolic material used. In an example, an optimal volume of embolic material can be higher when using softer embolic material and lower when using harder embolic material, or vice versa.

In an example, an optimal amount (or an optimal range of amounts) of embolic material or members can be expressed as a length, especially for a longitudinal embolic member or longitudinal series of embolic members which is dispensed through a lumen in a linear manner. In an example, an optical amount (or an optimal range of amounts) of embolic material or members can be expressed as a length of one or more embolic members which are longitudinally delivered through a catheter. In an example, an optimal amount (or an optimal range of amounts) of embolic material or members can be expressed as a number, especially for a plurality of separate and/or discrete embolic members which are dispensed through a lumen. In an example, an optical amount (or an optimal range of amounts) of embolic material or members can be expressed as a number of separate embolic members which are delivered through a catheter.

In an example, embolic material or members inserted into an aneurysm can be biocompatible yarn or fabric. In an example, embolic material or members inserted into an aneurysm can be blobs of gel. In an example, embolic material or members inserted into an aneurysm can be embolic coils. In an example, embolic material or members inserted into an aneurysm can be embolic gel which solidifies after insertion. In an example, embolic material or members inserted into an aneurysm can be embolic glue. In an example, embolic material or members inserted into an aneurysm can be embolic liquid which solidifies after insertion.

In an example, embolic material or members inserted into an aneurysm can be fiber strips. In an example, embolic material or members inserted into an aneurysm can be flexible wires. In an example, embolic material or members inserted into an aneurysm can be hydrogels. In an example, embolic material or members inserted into an aneurysm can be mesh ribbon. In an example, embolic material or members inserted into an aneurysm can be micro-beads. In an example, embolic material or members inserted into an aneurysm can be microscale mesh spheres.

In an example, embolic material or members inserted into an aneurysm can be microspheres. In an example, embolic material or members inserted into an aneurysm can be microsponges. In an example, embolic material or members inserted into an aneurysm can be stream of paste which solidifies after insertion. In an example, embolic material can comprise a shredded musical score, wherein a person can have a catchy tune stuck in their head. In an example, embolic material or members can be selected from the group consisting of: biocompatible yarn or fabric; blobs of gel; embolic coils; embolic gel which solidifies after insertion; embolic glue; embolic liquid which solidifies after insertion; fiber strips; flexible wires; hydrogels; mesh ribbon; micro-beads; microscale mesh spheres; microspheres; microsponges; stream of paste which solidifies after insertion; and string-of-pearls embolic structure (e.g. a plurality of embolic members connected by a string, filament, wire, or micro-chain). In an example, embolic material or members inserted into an aneurysm can be a string-of-pearls embolic structure (e.g. a plurality of embolic members inserted into an aneurysm connected by a string, filament, wire, or micro-chain).

In an example, embolic members can adhere to each other. In an example, embolic members can adhere to each other after they are inserted into a flexible net, mesh, bag, liner, or stent so that they are less likely to escape out of holes in the flexible net, mesh, bag, liner, or stent. In an example, embolic members can adhere to each other after they are inserted into an aneurysm so that they are less likely to protrude out of the aneurysm into the parent vessel. In an example, embolic members or material can have a first level of adhesion (or stickiness) before they are inserted into an aneurysm and have a second level of adhesion (or stickiness) after they are inserted into the aneurysm, wherein the second level is greater than the first level. In an example, embolic members or material can be changed from a first level of adhesion (or stickiness) to a second level of adhesion (or stickiness) by a means selected from the group consisting of: exposure to blood; exposure to body thermal energy; selective application of a chemical substance by a provider and/or device operator; selective application of electromagnetic energy by a provider and/or device operator; selective application of light energy in a selected wavelength by a provider and/or device operator; and selective application of thermal energy by a provider and/or device operator.

In an example, embolic members or material can be fused, congealed, stuck, or adhered together after insertion by selective intrasacular application of a chemical substance by a provider and/or device operator. In an example, embolic members or material can be fused, congealed, stuck, or adhered together after insertion by selective intrasacular application of electromagnetic energy by a provider and/or device operator. In an example, embolic members or material can be fused, congealed, stuck, or adhered together after insertion by selective intrasacular application of light energy in a selected wavelength by a provider and/or device operator. In an example, embolic members or material can be fused, congealed, stuck, or adhered together after insertion by selective intrasacular application of thermal energy by a provider and/or device operator.

In an example, a string-of-pearls embolic structure which is inserted into an aneurysm can comprise a plurality of embolic members (e.g. microsponges, microspheres, beads, or hydrogels) which are connected by one or more longitudinal flexible members (e.g. filaments, strings, threads, fibers, sutures, yarns, coils, or wires), wherein the surfaces of the embolic members have microscale (or nanoscale) hook-and-eye structures which cause the embolic members to stick to each other upon contact. This can help to prevent the embolic members from protruding out of the aneurysm. In an example, the embolic members can be separated from each other during delivery through a lumen so that they do not bunch together and clog the lumen, but can come into engaging contact with each other once they exit the lumen into the aneurysm.

In an example, the delivery of an optimal amount of embolic material or an optimal number of embolic members can be partially or fully automated based on an amount (or range of amounts) which is estimated using the above methodology. In an example, a device can automatically control the amount of embolic material and/or number of embolic members inserted into an aneurysm in order to insert an optimal amount of embolic material or an optimal number of embolic members. In an example, a device can automatically pump a flow of liquid or gelatinous embolic material into an aneurysm until the optimal amount of embolic material has been dispensed. In an example, a device can automatically push a series of longitudinal embolic members (such as coils) into an aneurysm until the optimal amount or number of embolic members has been dispensed. In an example, a device can automatically deliver a plurality of embolic members into an aneurysm until the optimal number of embolic members has been dispensed.

In an example, part of the implementation of these methods can be done with an embolic delivery control device which measures and controls the insertion of embolic material or members into an aneurysm so that the optimal amount of embolic material or members is inserted into the aneurysm. In an example, an embolic delivery control device for a longitudinal embolic member or series of longitudinal members (such as coils) can measure and control the length or number of embolic members inserted into the aneurysm. In an example, an embolic delivery control device to deliver an optimal length or number of longitudinal embolic members into an aneurysm can push a desired length or number of embolic members through a lumen into an aneurysm. In an example, this device can selectively cut, melt, snap, or segment an otherwise continuous length of embolic material (such as a coil) when the optimal length of the material has been inserted into an aneurysm.

In an example, an embolic delivery control device for a liquid or gelatinous embolic material can measure and control the flow of this embolic material into an aneurysm. In an example, an embolic delivery control device for a liquid or gelatinous embolic material can comprise a pump. In an example, an embolic delivery control device for a liquid or gelatinous embolic material can be selected from the group consisting of: axial pump, biochemical pump, biological pump, centrifugal pump, convective pump, diffusion pump, dispensing pump, effervescent pump, elastomeric pump, electrodiffusion pump, electrolytic pump, electromechanical pump, electroosmotic pump, fixed-occlusion peristaltic pump, gravity feed pump, helical pump, hose-type peristaltic pump, hydrolytic pump, In various examples, infusion pump, mechanical screw-type pump, Micro Electrical Mechanical System (MEMS) pump, micro pump, multiple-roller peristaltic pump, osmotic pump, peristaltic pump, piezoelectric pump, pulsatile pump, rotary pump, spring-loaded roller pump, tube-type peristaltic pump, and vapor pressure pump.

In an example, a liquid or gelatinous embolic material can be selected from the group consisting of: 2-octyl cyanoacrylate; ethyl-2-cyanoacrylate; methyl 2-cyanoacrylate; and n-butyl cyanoacrylate. In an example, a liquid or gelatinous embolic material can be selected from the group consisting of: acrylamide-based hydrogel; acrylic-acid-based hydrogel; agar; alginate-based hydrogel; carboxymethyl cellulose; cellulose; chitin; chitosan; collagen; copolymeric hydrogel; gellan; gum arabic; heparin; homopolymeric hydrogel; hyaluronan; hydrocolloid hydrogel; methyl cellulose; multipolymer interpenetrating polymeric hydrogel; pectin; pluronic-acid-based hydrogel; polyacrylic-acid-based hydrogel; polypeptide-based; polyurethane-based; poly-vinyl-alcohol-based hydrogel; starch; superabsorbent hydrogel; superporous hydrogel; and xanthan.

In an example, guidance concerning the optimal amount of embolic material or members can be partially, but not fully, automated. In an example, an embolic delivery control device can track and display the cumulative amount of embolic material or members which is being inserted into an aneurysm during a procedure. In an example, an embolic delivery control device can notify a provider and/or device operator in real time (e.g. with a visual, auditory, or tactile signal) as the cumulative amount of inserted embolic material or members is approaching the optimal amount. In an example, an embolic delivery control device can notify a provider and/or device operator in real time (e.g. with a visual, auditory, or tactile signal) as the cumulative amount of inserted embolic material or members has reached the optimal amount. Example variations discussed in other portions of this disclosure or in priority-linked disclosures can also be applied to these examples where relevant, but are not repeated here in order to reduce narrative redundancy.

In an example, a method to create a device to occlude a cerebral aneurysm can comprise: receiving a 3D image of a cerebral aneurysm; and using the 3D image to create a flexible net, mesh, bag, liner, or stent, wherein the flexible net, mesh, bag, liner, or stent is configured to be inserted into the aneurysm and then filled with embolic material or members in order to occlude the aneurysm.

In an example, a method to create a device to occlude a cerebral aneurysm can comprise: receiving a 3D image of a cerebral aneurysm; creating a 3D model of the aneurysm based on the 3D image of the cerebral aneurysm; and then using the 3D model of the aneurysm to create a flexible net, mesh, bag, liner, or stent, wherein the flexible net, mesh, bag, liner, or stent is configured to be inserted into the aneurysm and filled with embolic material or members in order to occlude the aneurysm. In an example, a CAD system can be used in the process of creating a 3D model from the 3D image.

In an example, an in-vivo 3D image of a cerebral aneurysm can be created by a medical imaging method selected from the group consisting of: Computerized Tomography (CT), Computerized Tomography Angiography (CTA), Cone Beam Computed Tomography (CBCT), Conoscopic Holography (CH), Digital Subtraction Angiography (DSA), Magnetic Resonance Angiography (MRA), Magnetic Resonance Imaging (MRI), Maximum Intensity Projection (MIP), Medical Holographic Imaging (MHI), Micro Computerized Tomography (MCT), Positron Emission Tomography (PET), Tuned-Aperture Computed Tomography (TACT), and Ultrasound (U/S). In an example, an in-vivo 3D image of a cerebral aneurysm can be a digital image. In an example, an in-vivo 3D image of a cerebral aneurysm can be a volumetric image. In an example, an in-vivo 3D image can be constructed by digitally merging a plurality of 2D images from different perspectives or at different times. In an example, an in-vivo 3D image of a cerebral aneurysm can be created after injection of contrast media into a person's bloodstream.

In an example, a method of 3D printing can be selected from the group consisting of: digital light processing, extrusion printing, fused deposition modeling, material jetting, photopolymerisation, powder-based printing, selective laser sintering, and stereolithography. In an example, a material used to create a 3D model of an aneurysm can be selected from the group consisting of: acrylic styrene acrylonitrile, acrylonitrile butadiene styrene, alumide, carbon fiber, carbon nanotubes, conductive carbomorph, glycol modified version of polyethylene terephthalate, graphene, high impact polystyrene, nitinol, nylon, polyamide, polycarbonate, polyether ether ketone, polyethylene terephthalate, polyjet resin, polylactic acid, polypropylene, polyvinyl alcohol, sintered powdered metal, and SLA resin. In an example, a CAD system can be used in the process of creating a 3D model from the 3D image.

In an example, a flexible net, mesh, bag, liner, or stent can be made from a 3D model of an aneurysm by the following steps: covering (or coating) the outer surface of a 3D model of an aneurysm with a flexible material; letting the flexible material congeal, solidify, and/or cure; and separating the flexible material from the 3D model. In an example, a flexible net, mesh, bag, liner, or stent can be made from a 3D model of an aneurysm by the following steps: covering (or coating) the outer surface of a 3D model of an aneurysm with a flexible material; letting the flexible material congeal, solidify, and/or cure; separating the flexible material from the 3D model; and creating holes in the flexible material.

In an example, a flexible net, mesh, bag, liner, or stent can be made from a 3D model of an aneurysm by the following steps: covering (or coating) the outer surface of a 3D model of an aneurysm with a flexible material; exposing the flexible material to thermal or electromagnetic energy to congeal, solidify, and/or cure the flexible material; and separating the flexible material from the 3D model. In an example, a flexible net, mesh, bag, liner, or stent can be made from a 3D model of an aneurysm by the following steps: covering (or coating) the outer surface of a 3D model of an aneurysm with a flexible material; exposing the flexible material to thermal or electromagnetic energy to congeal, solidify, and/or cure the flexible material; and separating the flexible material from the 3D model; and creating holes in the flexible material.

In an example, a flexible net, mesh, bag, liner, or stent can be made from a 3D model of an aneurysm by the following steps: covering (or coating) the inner surface of a 3D model of an aneurysm with a flexible material; letting the flexible material congeal, solidify, and/or cure; and separating the flexible material from the 3D model. In an example, a flexible net, mesh, bag, liner, or stent can be made from a 3D model of an aneurysm by the following steps: covering (or coating) the inner surface of a 3D model of an aneurysm with a flexible material; letting the flexible material congeal, solidify, and/or cure; separating the flexible material from the 3D model; and creating holes in the flexible material.

In an example, a flexible net, mesh, bag, liner, or stent can be made from a 3D model of an aneurysm by the following steps: covering (or coating) the inner surface of a 3D model of an aneurysm with a flexible material; exposing the flexible material to thermal or electromagnetic energy to congeal, solidify, and/or cure the flexible material; and separating the flexible material from the 3D model. In an example, a flexible net, mesh, bag, liner, or stent can be made from a 3D model of an aneurysm by the following steps: covering (or coating) the inner surface of a 3D model of an aneurysm with a flexible material; exposing the flexible material to thermal or electromagnetic energy to congeal, solidify, and/or cure the flexible material; and separating the flexible material from the 3D model; and creating holes in the flexible material.

In an example, a flexible material can be selected from the group consisting of: Dacron, ethylene tetrafluoroethylene, gold, latex, nylon, perfluoroalkoxy, platinum, polyacetal, polyamide, polyanhydride, polycaprolactone, polycarbonate (urethane), polydimethylsiloxane, polyester, polyether ether ketone, polyether polyamide copolymer, polyethylene terephthalate, polylactide, polyolefin, polyphosphazene, polyphosphoester, polypropylene, polytetrafluoroethene, polyurethane, polyvinyl chloride, polywanacrakur, silicone, and Teflon. In an example, a flexible material can be selected from the group consisting of: chromium, iridium, molybdenum, nickel, nitinol, platinum, steel, titanium, and tungsten.

In an example, a flexible net, mesh, bag, liner, or stent can be made from one or more materials selected from the group consisting of: Dacron, ethylene tetrafluoroethylene, gold, latex, nylon, perfluoroalkoxy, platinum, polyacetal, polyamide, polyanhydride, polycaprolactone, polycarbonate (urethane), polydimethylsiloxane, polyester, polyether ether ketone, polyether polyamide copolymer, polyethylene terephthalate, polylactide, polyolefin, polyphosphazene, polyphosphoester, polypropylene, polytetrafluoroethene, polyurethane, polyvinyl chloride, polywanacrakur, silicone, and Teflon. In an example, a flexible net, mesh, bag, liner, or stent can be made from one or more materials selected from the group consisting of: chromium, iridium, molybdenum, nickel, nitinol, platinum, steel, titanium, and tungsten.

In an example, a flexible material can be applied or to the surface a 3D model of aneurysm by a method selected from the group consisting of: spraying; squirting; dipping; dripping; printing; melting; pouring; and brushing. In an example, holes can be made in flexible material using light energy. In an example, a laser can be used to make holes in flexible material. In an example, holes can be made in flexible material using thermal energy. In an example, holes can be created in flexible material by melting. In an example, a flexible material can be conformed to the surface a 3D model of aneurysm by a method selected from the group consisting of: annealing, braiding, heat-setting, weaving, and wrapping. In example, holes can be formed in a flexible net, mesh, bag, liner, or stent by a weaving or braiding process.

In an example, a method to create a device to occlude a cerebral aneurysm can comprise: receiving a 3D image of a cerebral aneurysm; creating a 3D model of the aneurysm based on the 3D image of the aneurysm, wherein the 3D model is made with a first material, and wherein this first material has a first level of flexibility or elasticity; and using the 3D model to create a flexible net, mesh, bag, liner, or stent from a second material, wherein this second material has a second level of flexibility or elasticity, wherein the second level is greater than the first level, and wherein the flexible net, mesh, bag, liner, or stent is configured to be inserted into the aneurysm and filled with embolic material or members in order to occlude the aneurysm.

In an example, a digital 3D image of a particular cerebral aneurysm can be created using a medical imaging modality such as CT or MRI. Then, this digital 3D image can be used to 3D print a 3D model of this particular aneurysm using a first material that is relatively rigid. Then, a second material in a liquid or gelatinous state can be sprayed, printed, or otherwise applied to the surface of the 3D model. Then the second material can be exposed to selected thermal, electromagnetic, and/or light energy to solidify, congeal, and/or cure the second material. Alternatively, the second material can solidify, congeal, or cure over time even without exposure to selected thermal, electromagnetic, and/or light energy. Then the second material is separated from the 3D model. In an example, this separation can be done by peeling the second material away from the 3D model. In an example, this separation can be done by dissolving the 3D model. Alternatively, the second material can be separated from the 3D model before the second material is solidified, congealed, or cured. In an example, holes can be made in the second material to create a flexible net, mesh, bag, liner, or stent for insertion into the aneurysm. In an example, holes can be made using a laser.

In an example, a first material can be selected from the group consisting of: acrylic styrene acrylonitrile, acrylonitrile butadiene styrene, alumide, carbon fiber, carbon nanotubes, conductive carbomorph, glycol modified version of polyethylene terephthalate, graphene, high impact polystyrene, nitinol, nylon, polyamide, polycarbonate, polyether ether ketone, polyethylene terephthalate, polyjet resin, polylactic acid, polypropylene, polyvinyl alcohol, sintered powdered metal, and SLA resin. In an example, a second material can be selected from the group consisting of: Dacron, ethylene tetrafluoroethylene, latex, nylon, perfluoroalkoxy, polycarbonate urethane, polydimethylsiloxane, polyester, polyether ether ketone, polyether polyamide copolymer, polyethylene terephthalate, polyolefin, polypropylene, polytetrafluoroethene, polyurethane, polyvinyl chloride, silicone, and Teflon. In an example, the second material can be selected from the group consisting of: chromium, iridium, molybdenum, nickel, nitinol, platinum, steel, titanium, and tungsten.

In an example, the flexible net, mesh, bag, liner, or stent which results from these steps can be compressed (e.g. folded, rolled, squeezed, pleated, or shrunk) into a first configuration for delivery through a lumen into a cerebral aneurysm. In an example, a flexible net, mesh, bag, liner, or stent can be expanded into a second configuration within the cerebral aneurysm by insertion of embolic material or members into the flexible net, mesh, bag, liner, or stent. In an example, the second configuration of the flexible net, mesh, bag, liner, or stent will have the morphology of this particular aneurysm because the flexible net, mesh, bag, liner, or stent was created by using a 3D model of this particular aneurysm. This can reduce or eliminate gaps in the aneurysm for more complete aneurysm occlusion and prevention of blood flow into the aneurysm.

In an example, a method for occluding a cerebral aneurysm can comprise: receiving a 3D image of a cerebral aneurysm; creating a flexible net, mesh, bag, liner, or stent whose size and shape is based on the 3D image; inserting the flexible net, mesh, bag, liner, or stent into the cerebral aneurysm; and filling the flexible net, mesh, bag, liner, or stent within the cerebral aneurysm with embolic material or members. In an example, a method for occluding a cerebral aneurysm can comprise: creating a flexible mesh structure, wherein this mesh structure has a proximal portion and distal portion; inserting the flexible mesh structure into a cerebral aneurysm; positioning the flexible mesh structure over the neck of the cerebral aneurysm; compressing flexible mesh structure by moving the distal portion toward the proximal portion; and inserting embolic material or members into the portion of the cerebral aneurysm which is distal to the flexible mesh structure.

In an example, a flexible mesh structure can have a first configuration with a first shape and a second configuration with a second shape. In an example, a flexible mesh structure can be changed from its first configuration to its second configuration by moving its distal portion toward its proximal portion, or vice versa. In an example, the first shape can be a three-dimensional convex shape. In an example, the first shape (of a flexible mesh structure in its first configuration) can be selected from the group consisting of: sphere; ellipsoid; ovaloid; apple shape; barrel shape; pear shape; and egg shape. In an example, the second shape (of a flexible mesh structure in its second configuration) can be selected from the group consisting of: (inverted) dome surface, (inverted) umbrella shape, bowl shape, elliptic paraboloid, hemispherical surface, paraboloid, and spherical cap. In an example, the distal portion of a flexible mesh structure can be moved toward the proximal portion of a flexible mesh structure by a means selected from the group consisting of: pulling the distal portion with a wire, filament, string, or chain; electromagnetic attraction between the distal portion and the proximal portion; activation of a microscale electromagnetic actuator such as a MEMS actuator; moving the distal portion with a micro-pneumatic mechanism; moving the distal portion a micro-hydraulic mechanism; and pushing a proximal portion with a wire, catheter, or other resilient member.

In an example, a method for occluding a cerebral aneurysm can comprise: (a) inserting a flexible net, mesh, bag, liner, or stent into the cerebral aneurysm; wherein the flexible net, mesh, bag, liner, or stent has a first configuration with a first shape; and wherein the first shape is selected from the group consisting of sphere, ellipsoid, ovaloid, apple shape, barrel shape, pear shape, and egg shape; (b) compressing the flexible net, mesh, bag, liner, or stent into a second configuration within the cerebral aneurysm; wherein the flexible net, mesh, bag, liner, or stent has a second shape in the second configuration; and wherein the second shape is selected from the group consisting of (inverted) dome surface, (inverted) umbrella shape, bowl shape, elliptic paraboloid, hemispherical surface, paraboloid, and spherical cap; and (c) inserting embolic material or members into the portion of the aneurysm sac which is distal to the flexible net, mesh, bag, liner, or stent. In an example, a flexible net, mesh, bag, liner, or stent can be compressed within an aneurysm by a means selected from the group consisting of: pulling a distal portion of the flexible net, mesh, bag, liner, or stent with a wire, filament, string, or chain; electromagnetic attraction between distal and proximal portions of the flexible net, mesh, bag, liner, or stent; activation of a microscale electromagnetic actuator such as a MEMS actuator; moving a distal portion of the flexible net, mesh, bag, liner, or stent with a micro-pneumatic mechanism; moving the distal portion of the flexible net, mesh, bag, liner, or stent with a micro-hydraulic mechanism; and pushing a proximal portion of the flexible net, mesh, bag, liner, or stent with a wire, catheter, or other resilient member.

In an example, a method for occluding a cerebral aneurysm can comprise: inserting a net, mesh, bag, liner, or stent into an cerebral aneurysm, wherein the net, mesh, bag, liner, or stent self-expands into a toroidal shape within the aneurysm sac; inserting embolic material or members into the distal portion of the aneurysm sac through an opening in the net, mesh, bag, liner, or stent; and then closing the opening in the net, mesh, bag, liner, or stent. In an example, a method for occluding a cerebral aneurysm can comprise: inserting a net, mesh, bag, liner, or stent into an cerebral aneurysm, wherein the net, mesh, bag, liner, or stent self-expands into a spherical or ellipsoidal shape within the aneurysm sac; inserting embolic material or members into the distal portion of the aneurysm sac through an opening in the net, mesh, bag, liner, or stent; and then closing the opening in the net, mesh, bag, liner, or stent. In an example, a method for occluding a cerebral aneurysm can comprise: inserting a net, mesh, bag, liner, or stent into an cerebral aneurysm, wherein the net, mesh, bag, liner, or stent self-expands into a bowl or paraboloid shape within the aneurysm sac; inserting embolic material or members into the distal portion of the aneurysm sac through an opening in the net, mesh, bag, liner, or stent; and then closing the opening in the net, mesh, bag, liner, or stent.

In an example, the above processes can result in a flexible net, mesh, bag, liner, or stent whose size and shape are customized to the size and shape of a particular aneurysm. In an example, a flexible net, mesh, bag, liner, or stent can further comprise one or more fiducial markers which are visible in medical imaging so that a provider can adjust the orientation of the flexible net, mesh, bag, liner, or stent (before or during expansion) to match the orientation of the aneurysm. In an example, these fiducial markers can be radio-opaque, radiolucent, echogenic, CT visible, and/or MR visible. In an example, proper alignment of the flexible net, mesh, bag, liner, or stent within the aneurysm sac can be computer assisted in real time in a manner analogous to computer-assisted docking between two space craft. In an example, a fiducial marker can comprise one (or more) metal spheres (or other metal pieces or particles) which are inserted (or embedded) into a flexible net, mesh, bag, liner, or stent. In an example, one or more fiducial markers can be located at non-axial locations (e.g. not along the longitudinal axis) of the flexible net, mesh, bag, liner, or stent such that these fiducial markers move when the flexible net, mesh, bag, liner, or stent is rotated around its longitudinal axis.

In an example, one or more fiducial markers can be created by selectively adding radio-opaque, radiolucent, echogenic, CT visible, and/or MR visible material to selected locations of a flexible net, mesh, bag, liner, or stent. In an example, radio-opaque, radiolucent, echogenic, CT visible, and/or MR visible material can be added to selected locations of a flexible net, mesh, bag, liner, or stent before the flexible net, mesh, bag, liner, or stent is solidified, congealed, and/or cured. Radio-opaque, radiolucent, echogenic, CT visible, and/or MR visible material can be selectively added to selected locations of a flexible net, mesh, bag, liner, or stent while the primary material of the flexible net, mesh, bag, liner, or stent is being sprayed or otherwise applied to the surface of a 3D model. In an example, radio-opaque, radiolucent, echogenic, CT visible, and/or MR visible material can be selectively added to two (radial) locations around a perimeter of a flexible net, mesh, bag, liner, or stent. In an example, radio-opaque, radiolucent, echogenic, CT visible, and/or MR visible material can be selectively added to four or more (radial) locations around a perimeter of a flexible net, mesh, bag, liner, or stent. In an example, different types of radio-opaque, radiolucent, echogenic, CT visible, and/or MR visible material can be added to different locations of a flexible net, mesh, bag, liner, or stent for more accurate in-vivo tracking of the orientation of the flexible net, mesh, bag, liner, or stent during medical imaging.

In an example, virtual fiducial markers can be added to identify selected locations on a cerebral aneurysm during medical imaging. The locations of virtual fiducial markers can be matched to, and moved with, actual locations on the cerebral aneurysm by dynamic computer-automated pattern identification using augmented reality (AR) techniques. A computer can keep track of the location and orientation of the aneurysm based on observed geometry in real time and keep virtual fiducial markers at desired locations with respect to the aneurysm. In an example, actual radio-opaque, radiolucent, echogenic, CT visible, and/or MR visible fiducial markers on a flexible net, mesh, bag, liner, or stent can be manually aligned with virtual fiducial markers on the aneurysm in order to properly align a custom-formed flexible net, mesh, bag, liner, or stent to the orientation of the aneurysm. This alignment can be done before or during expansion of the flexible net, mesh, bag, liner, or stent. In an example, alignment of actual radio-opaque, radiolucent, echogenic, CT visible, and/or MR visible fiducial markers on the flexible net, mesh, bag, liner, or stent with the virtual fiducial markers on the aneurysm can be partially (or completely) automated with the help of a computer guidance system and a plurality of directional/rotational actuators.

In an example, a method to create a device to occlude a cerebral aneurysm can comprise: receiving a 3D image of a cerebral aneurysm; creating a 3D model of the aneurysm based on the 3D image of the cerebral aneurysm; applying a first material to a first area of the surface of the 3D model of the aneurysm; and applying a second material to a second area of the surface of the 3D model of the aneurysm, wherein the first material has a first level of radio-opaqueness, radiolucence, or echogenicity, wherein the second material has a second level of radio-opaqueness, radiolucence, or echogenicity, and wherein the second level is greater than the first level. In an example, a method to create a device to occlude a cerebral aneurysm can comprise: receiving a 3D image of a cerebral aneurysm; creating a 3D model of the aneurysm based on the 3D image of the cerebral aneurysm; applying a first material to first locations on the surface of the 3D model of the aneurysm; and applying a second material to second locations on the surface of the 3D model of the aneurysm, wherein the first material has a first level of radio-opaqueness, radiolucence, or echogenicity, wherein the second material has a second level of radio-opaqueness, radiolucence, or echogenicity, and wherein the second level is greater than the first level.

In an example, a method to create a device to occlude a cerebral aneurysm can comprise: receiving a 3D image of a cerebral aneurysm; creating a 3D model of the aneurysm based on the 3D image of the cerebral aneurysm; spraying, coating, printing, or otherwise applying a first material to the 3D model of the aneurysm; and spraying, coating, printing, or otherwise applying a second material to selected locations on the 3D model of the aneurysm, wherein the second material is radio-opaque, radiolucent, or echogenic. In an example, the selected locations can be distributed around a lateral circumference of the 3D model. In an example, the selected locations can comprise two locations on a lateral circumference of the 3D model. In an example, the selected locations can comprise four or more locations on a lateral circumference of the 3D model.

In an example, a method to create a device to occlude a cerebral aneurysm can comprise: receiving a 3D image of a cerebral aneurysm; creating a 3D model based on the 3D image; and conforming a plurality of wires around the 3D model in order to create a custom-shaped convex flexible wire mesh which is configured to be inserted into that particular aneurysm and then filled with embolic material or members. In an example, wires can be made from one or more materials selected from the group consisting of: chromium, iridium, molybdenum, nickel, nitinol, platinum, steel, titanium, and tungsten. In an example, wires can be conformed to a 3D model by wrapping, coiling, weaving, braiding, spooling, 3D printing, annealing, or melting. In an example, thermal energy can be applied to wires as they are being conformed to a 3D model. In an example, thermal energy can be applied to wires after they have been conformed to a 3D model. In an example, wires can be heat-set on a 3D model so that they are biased toward returning to the shape that they have on the 3D model.

In an example, a 3D image of a cerebral aneurysm can be created by a medical imaging method selected from the group consisting of: Computerized Tomography (CT), Computerized Tomography Angiography (CTA), Cone Beam Computed Tomography (CBCT), Conoscopic Holography (CH), Digital Subtraction Angiography (DSA), Magnetic Resonance Angiography (MRA), Magnetic Resonance Imaging (MRI), Maximum Intensity Projection (MIP), Medical Holographic Imaging (MHI), Micro Computerized Tomography (MCT), Positron Emission Tomography (PET), Tuned-Aperture Computed Tomography (TACT), and Ultrasound (U/S).

In an example, a 3D image of a cerebral aneurysm can be a digital image. In an example, a 3D image of a cerebral aneurysm can be a volumetric image. In an example, a 3D image can be constructed by digitally merging a plurality of 2D images from different perspectives or at different times. In an example, a 3D image of a cerebral aneurysm can be created after injection of contrast media into a person's bloodstream. In an example, a CAD system can be used in the process of creating a 3D model or 3D mandrel from a 3D image. In an example, 3D printing can be used to create a 3D model or 3D mandrel from a 3D image.

In an example, a method to create a device to occlude a cerebral aneurysm can comprise: receiving a 3D image of a cerebral aneurysm; creating a 3D mandrel based on the 3D image; and coiling or wrapping wires or coils around the 3D mandrel in order to create a custom-shaped convex flexible wire mesh which is configured to be inserted into that particular aneurysm and then filled with embolic material or members. In an example, a method to create a device to occlude a cerebral aneurysm can comprise: receiving a 3D image of a cerebral aneurysm; creating a 3D model or 3D mandrel based on the 3D image; and wrapping, weaving, braiding, melting, shrinking, or otherwise conforming wires around the 3D model or 3D mandrel in order to create a custom-shaped convex flexible wire mesh which is configured to be inserted into the aneurysm and then filled with embolic material or members.

In an example, wires can be annealed on a 3D model of an aneurysm or a 3D mandrel based on the aneurysm in order to create a custom-shaped convex flexible wire mesh to be inserted into the aneurysm. In an example, wires can be braided on a 3D model of an aneurysm or a 3D mandrel based on the aneurysm in order to create a custom-shaped convex flexible wire mesh to be inserted into the aneurysm. In an example, wires can be coiled around a 3D model of an aneurysm or a 3D mandrel based on the aneurysm in order to create a custom-shaped convex flexible wire mesh to be inserted into the aneurysm. In an example, wires can be heat set on a 3D model of an aneurysm or a 3D mandrel based on the aneurysm in order to create a custom-shaped convex flexible wire mesh to be inserted into the aneurysm. In an example, wires can be heated on a 3D model of an aneurysm or a 3D mandrel based on the aneurysm in order to create a custom-shaped convex flexible wire mesh to be inserted into the aneurysm. In an example, wires can be wrapped around a 3D model of an aneurysm or a 3D mandrel based on the aneurysm in order to create a custom-shaped convex flexible wire mesh to be inserted into the aneurysm. In an example, rapping can be done by snoopdogg. In an example, wires can be woven around a 3D model of an aneurysm or a 3D mandrel based on the aneurysm in order to create a custom-shaped convex flexible wire mesh to be inserted into the aneurysm.

In an example, a custom-shaped convex flexible wire mesh which is created around a 3D model or 3D mandrel can further comprise one or more radio-opaque, radiolucent, echogenic, CT visible, and/or MR visible components. In an example, during insertion and expansion of the custom-shaped convex flexible wire mesh in an aneurysm sac, these one or more radio-opaque, radiolucent, echogenic, CT visible, and/or MR visible components can be aligned with selected locations on the aneurysm sac to ensure that the custom-shaped wire mesh is properly aligned with the aneurysm. In an example, Augmented Reality (AR) can be used to create virtual fiducial markers at selected locations on the aneurysm and the radio-opaque, radiolucent, echogenic, CT visible, and/or MR visible components on the custom-shaped convex flexible wire mesh can be aligned with these virtual fiducial markets to ensure that the mesh is properly aligned with the aneurysm.

In an example, a method to occlude a cerebral aneurysm can comprise: receiving a 3D image of a cerebral aneurysm; and analyzing the 3D image to estimate an optimal amount of embolic material to be inserted into a flexible net, mesh, bag, liner, or stent within the aneurysm in order to occlude the aneurysm. In an example, a 3D image of the cerebral aneurysm can be created by a medical imaging method selected from the group consisting of: Computerized Tomography (CT), Computerized Tomography Angiography (CTA), Cone Beam Computed Tomography (CBCT), Conoscopic Holography (CH), Digital Subtraction Angiography (DSA), Magnetic Resonance Angiography (MRA), Magnetic Resonance Imaging (MRI), Maximum Intensity Projection (MIP), Medical Holographic Imaging (MHI), Micro Computerized Tomography (MCT), Positron Emission Tomography (PET), Tuned-Aperture Computed Tomography (TACT), and Ultrasound (U/S).

In an example, an optimal amount of embolic material can be calculated by estimating the total interior volume of the aneurysm based on the 3D image of the aneurysm and then subtracting the volume of the perimeter layer of the flexible net, mesh, bag, liner, or stent. In an example, estimation of the optimal amount of embolic material can depend on one or more factors selected from the group consisting of: general aneurysm shape or type; aneurysm size; aneurysm location; aneurysm rupture status; type of embolic material; shape of embolic material; size of embolic material; softness and/or compressibility of embolic material; parent vessel shape; parent vessel location; patient demographic information; and patient medical history.

In an example, an embolic material can comprise embolic coils or ribbons. In an example, an embolic material can comprise hydrogels or other gelatinous material. In an example, an embolic material can comprise a string-of-pearls structure (i.e. a plurality of embolic members connected by a string, filament, wire, or micro-chain). In an example, a flow of liquid or gelatinous embolic material can be (automatically) pumped into the flexible net, mesh, bag, liner, or stent until the optimal amount of embolic material has been dispensed. In an example, embolic members can be (automatically) delivered into the flexible net, mesh, bag, liner, or stent until the optimal amount of embolic members has been dispensed.

In an example, a method to occlude a cerebral aneurysm can comprise: receiving a 3D image of a cerebral aneurysm; and analyzing the 3D image to estimate an optimal amount of embolic material to be inserted into the aneurysm in order to occlude the aneurysm. In an example, a 3D image of the cerebral aneurysm can be created by a medical imaging method selected from the group consisting of: Computerized Tomography (CT), Computerized Tomography Angiography (CTA), Cone Beam Computed Tomography (CBCT), Conoscopic Holography (CH), Digital Subtraction Angiography (DSA), Magnetic Resonance Angiography (MRA), Magnetic Resonance Imaging (MRI), Maximum Intensity Projection (MIP), Medical Holographic Imaging (MHI), Micro Computerized Tomography (MCT), Positron Emission Tomography (PET), Tuned-Aperture Computed Tomography (TACT), and Ultrasound (U/S).

In an example, an optimal amount of embolic material can be calculated by estimating the total interior volume of the aneurysm based on the 3D image of the aneurysm. In an example, estimation of the optimal amount of embolic material can depend on one or more factors selected from the group consisting of: general aneurysm shape or type; aneurysm size; aneurysm location; aneurysm rupture status; type of embolic material; shape of embolic material; size of embolic material; softness and/or compressibility of embolic material; parent vessel shape; parent vessel location; patient demographic information; and patient medical history.

In an example, an embolic material can comprise embolic coils or ribbons. In an example, an embolic material can comprise hydrogels or other gelatinous material. In an example, an embolic material can comprise a string-of-pearls structure (i.e. a plurality of embolic members connected by a string, filament, wire, or micro-chain). In an example, a flow of liquid or gelatinous embolic material can be (automatically) pumped into the cerebral aneurysm until the optimal amount of embolic material has been dispensed. In an example, embolic members can be (automatically) delivered into the cerebral aneurysm until the optimal amount of embolic members has been dispensed.

In an example, a method to create a device to occlude a cerebral aneurysm can comprise: receiving a 3D image of a cerebral aneurysm; creating a 3D model or 3D mandrel based on the 3D image; and wrapping, weaving, braiding, melting, shrinking, or otherwise conforming wires around the 3D model or 3D mandrel in order to create a custom-shaped convex flexible wire mesh which is configured to be inserted into the aneurysm. In an example, embolic material can be inserted into custom-shaped convex flexible wire mesh after the mesh has been inserted into the cerebral aneurysm.

I claim:

1. A method to occlude a cerebral aneurysm comprising:
receiving a 3D image of a cerebral aneurysm; and
analyzing the 3D image to estimate an optimal amount of embolic material to be inserted into a flexible net, mesh, bag, liner, or stent within the aneurysm in order to occlude the aneurysm.

2. The method in claim 1 wherein the 3D image of the cerebral aneurysm is created by a medical imaging method selected from the group consisting of: Computerized Tomography (CT), Computerized Tomography Angiography (CTA), Cone Beam Computed Tomography (CBCT), Conoscopic Holography (CH), Digital Subtraction Angiography (DSA), Magnetic Resonance Angiography (MRA), Magnetic Resonance Imaging (MRI), Maximum Intensity Projection (MIP), Medical Holographic Imaging (MHI), Micro Computerized Tomography (MCT), Positron Emission Tomography (PET), Tuned-Aperture Computed Tomography (TACT), and Ultrasound (U/S).

3. The method in claim 1 wherein the optimal amount of embolic material is calculated by estimating the total interior volume of the aneurysm based on the 3D image of the aneurysm and then subtracting the volume of the perimeter layer of the flexible net, mesh, bag, liner, or stent.

4. The method in claim 1 wherein estimation of the optimal amount of embolic material depends on one or more factors selected from the group consisting of: general aneurysm shape or type; aneurysm size; aneurysm location; aneurysm rupture status; type of embolic material; shape of embolic material; size of embolic material; softness and/or compressibility of embolic material; parent vessel shape; parent vessel location; patient demographic information; and patient medical history.

5. The method in claim 1 wherein the embolic material comprises embolic coils or ribbons.

6. The method in claim 1 wherein the embolic material comprises hydrogels or other gelatinous material.

7. The method in claim 1 wherein the embolic material comprises a string-of-pearls structure (i.e. a plurality of embolic members connected by a string, filament, wire, or micro-chain).

8. The method in claim 1 wherein a flow of liquid or gelatinous embolic material is pumped into the flexible net, mesh, bag, liner, or stent until the optimal amount of embolic material has been dispensed.

9. The method in claim 1 wherein embolic members are delivered into the flexible net, mesh, bag, liner, or stent until the optimal amount of embolic members has been dispensed.

10. A method to occlude a cerebral aneurysm comprising:
receiving a 3D image of a cerebral aneurysm; and
analyzing the 3D image to estimate an optimal amount of embolic material to be inserted into the aneurysm in order to occlude the aneurysm.

11. The method in claim 10 wherein the 3D image of the cerebral aneurysm is created by a medical imaging method selected from the group consisting of: Computerized Tomography (CT), Computerized Tomography Angiography (CTA), Cone Beam Computed Tomography (CBCT), Conoscopic Holography (CH), Digital Subtraction Angiography (DSA), Magnetic Resonance Angiography (MRA), Magnetic Resonance Imaging (MRI), Maximum Intensity Projection (MIP), Medical Holographic Imaging (MHI), Micro Computerized Tomography (MCT), Positron Emission Tomography (PET), Tuned-Aperture Computed Tomography (TACT), and Ultrasound (U/S).

12. The method in claim 10 wherein the optimal amount of embolic material is calculated by estimating the total interior volume of the aneurysm based on the 3D image of the aneurysm.

13. The method in claim 10 wherein estimation of the optimal amount of embolic material depends on one or more factors selected from the group consisting of: general aneurysm shape or type; aneurysm size; aneurysm location; aneurysm rupture status; type of embolic material; shape of embolic material; size of embolic material; softness and/or compressibility of embolic material; parent vessel shape; parent vessel location; patient demographic information; and patient medical history.

14. The method in claim 10 wherein the embolic material comprises embolic coils or ribbons.

15. The method in claim 10 wherein the embolic material comprises hydrogels or other gelatinous material.

16. The method in claim 10 wherein the embolic material comprises a string-of-pearls structure (i.e. a plurality of embolic members connected by a string, filament, wire, or micro-chain).

17. The method in claim 10 wherein a flow of liquid or gelatinous embolic material is pumped into the cerebral aneurysm until the optimal amount of embolic material has been dispensed.

18. The method in claim 10 wherein embolic members are delivered into the cerebral aneurysm until the optimal amount of embolic members has been dispensed.

19. A method to create a device to occlude a cerebral aneurysm comprising:
receiving a 3D image of a cerebral aneurysm;
creating a 3D model or 3D mandrel based on the 3D image; and
wrapping, weaving, braiding, melting, shrinking, or otherwise conforming wires around the 3D model or 3D mandrel in order to create a custom-shaped convex flexible wire mesh which is configured to be inserted into the aneurysm.

20. The method in claim 19 wherein embolic material is inserted into custom-shaped convex flexible wire mesh after the mesh has been inserted into the cerebral aneurysm.
